(19) United States
(12) Patent Application Publication
(10) Pub. No.: US 2014/0031897 A1
(43) Pub. Date: Jan. 30, 2014

(54) DEVICE FOR THE TREATMENT OF SKIN

(71) Applicant: Edelgard Liebl, Zelsheim/Elsass (FR)

(72) Inventor: Horst LIEBL, Zelsheim (FR)

(21) Appl. No.: 13/944,440

(22) Filed: Jul. 17, 2013

(30) Foreign Application Priority Data
Jul. 24, 2012 (DE) 10 2012 014 653.1

(51) Int. Cl. A61N 1/36

(52) U.S. Cl. CPC A61N 1/36
USPC 607/50

(57) ABSTRACT

A skin treatment device includes a roller rotatably mounted about its longitudinal axis and having needles protruding radially outwardly from its peripheral surface, and a device for establishing electrical contact between at least part of the needles and a voltage source having first and second poles, including first and second terminals capable of being electrically connected to the first and second poles, respectively, a first contacting zone electrically connected to first needles, a second contacting zone spaced and electrically insulated from the first contacting zone and electrically connected to second needles, the terminals and the contacting zones being configured such that when the first terminal is connected to the first contacting zone, the second terminal is likewise connected to the second contacting zone. The first and the second needles are each arranged in a row, the rows being separated from each other in a circumferential direction of the roller.

### CROSS REFERENCE TO RELATED APPLICATIONS

Applicant claims priority under 35 U.S.C. §119 of German Application No. 10 2012 014 653.1 filed Jul. 24, 2012, the disclosure of which is incorporated by reference.

### BACKGROUND OF THE INVENTION

1. Field of the Invention

The invention relates to a device for the treatment of skin, which device comprises a roller mounted for rotation about its longitudinal axis and comprising a number of needles protruding radially outwardly from its peripheral surface around the same, for the purpose of applying an electric potential to the skin, for example for the purpose of promoting the healing of wounds.

2. The Prior Art

Without going into the etiology of chronic or non-healing wounds in detail, it is a fact that the main cause of a slow or discontinued healing process is an insufficient blood supply to the region of a wound. In this case, the wound is only insufficiently, or no longer, supplied with oxygen and nutrient substances. There are also other causes of bacterial and/or toxic origin that hinder or even completely suppress the proliferation of wound cleansing cells and wound repairing cells. All of these causes lead to the breakdown of cell information by chemotactic and/or electrotactic processes and prevent the proliferation of cells for the generation of new tissue. Each wound, acute or chronic, leads to a breakdown of the bioelectric potentials (trans epithelial potentials—TEPs (Foulds and Barker [1, 19] (see “Literature”), i.e. to short-circuiting thereof (Kloth [2]). Most probably, a deficiency, a pH shift, or exsiccation of the interstitial electrolyte [3, 4] additionally prevents the activity of the ion pump for the cells in the vicinity of the wound. This is always activated after an injury and serves the purpose of generating a natural electromagnetic field and activates the gene expression of transforming growth factors (TGFs) and the proliferation of fibroblasts and other cells. These deficiency manifestations suppress the generation of a natural electromagnetic field [5].

The most important prerequisites for the management of chronic wounds include four components: a) tissue management, b) inflammation and infection control, c) moisture balance, and d) epithelization. In many cases, however, the wound healing process will stop, there is often closure of the wound, but in many cases amputation of the extremities concerned is unavoidable.

Theoretical considerations and measurements, apparatus, and devices used in practice have shown that chronic wounds can be positively influenced by means of electrostimulation to an extent that this can cause reclosure of the wound. Falanga et al. [6] have shown in vitro that the electrostimulation up-regulates the receptors of TGF-β on human dermal fibroblasts by a factor of six compared with control fibroblasts. Other investigations have shown that cells participating in wound healing migrate to the anode or cathode of an electric field (galvanotaxis) [7-17]. Most probably, cells react to electric fields through electrophoretic motion of proteins within the plasma membrane [18]. Likewise, the migration of keratinocytes is influenced by electric fields [14, 15]. As Rowley [19] and Wolcott et al. [20] have shown, a direct current electric field has a bacteriostatic effect. As a logical consequence, other research workers have been working on the use of silver-coated anodes [21-28] and have been able to show, in vitro and in vivo, that silver cations have a bacteriostatic and antibacterial effect. Junger et al. [29] have reported on an improvement in blood flow by increasing the capillary density by means of electric fields. In the case of 15 patients with leg ulcers, following a period of many months of standard treatment, the state of the ulcers improved on application of an electric field by 43.5% on average.

The drawback of all hitherto used wound treating devices utilizing positive and negative electrodes is that it is not possible to generate exactly defined currents to, and in, the wound. The underlying causes are diverse and will only be briefly outlined here:
a fluctuating moist milieu over and in the wound,
the distance of the position of the (counter) electrode from the electrode on the wound varies,
the electric resistance can be influenced by fluctuating skin moisture under the counterelectrode,
chronic wounds (though rarely) occurring above the heart meridian cannot be treated due to the fact that they might possibly be affected by electrical cardiac and cerebral activity.
   On account of the aforementioned problems, the devices of the prior art suffer from the drawback that they frequently fail to achieve satisfactory and reproducible results as regards wound treatment.

### SUMMARY OF THE INVENTION

It is thus an object of the present invention to provide a device for the treatment of skin, which makes it possible to apply, in a specific and reproducible manner, an electric field to the skin to be treated and thus to induce a flow of ions therein.

This object is successfully achieved with the device according to the invention. Preferred embodiments are described below. Accordingly, the invention relates to a device for the treatment of skin, which device comprises a roller mounted for rotation about its longitudinal axis and comprising a number of needles protruding radially outwardly from its peripheral surface around the same. Such devices in which the roller equipped with microneedles is rolled over the skin to be treated are known per se and are described in DE 10063634 A1 of the applicant. Concerning the basic construction of such a device, reference may also be made to EP 1764129 A1 of the applicant. The device described in said reference comprises a plurality of rollers equipped with needles. The needles piercing the skin during rotating of the roller(s) stimulate collagenesis and angiogenesis and promote regeneration of the skin and wound healing even in the absence of active substances. In addition, hypertrophic scars can be catabolized and atrophic scars built up. Due to the temporary formation of fine puncture channels, it is possible for active substances to be much more readily absorbed by the dermis due to easier passage thereof through the epidermal barrier.

The present invention constitutes a development of the aforementioned devices in that the latter are provided with means for making electrical contact between at least part of the needles and a voltage source. In more detail, the invention relates to a device for the treatment of skin, comprising a roller which is mounted for rotation about its longitudinal axis and on a peripheral surface of which a number of needles protrudes radially outwardly, and comprising means for establishing electrical contact between at least part of said needles and a voltage source, comprising a first terminal that is capable of being electrically connected to a first pole of said voltage source, a second terminal that is capable of being electrically connected to a second pole of said voltage source, a first contacting zone that is electrically connected to first needles, a second contacting zone that is spaced from, and electrically insulated from, said first contacting zone and that is electrically connected to second needles, said terminals and said contacting zones being configured such that when said first terminal is connected to said first contacting zone, said second terminal is connected to said second contacting zone, wherein said first and said second needles are each arranged in a row, said rows being separated from each other in a circumferential direction of said roller.

In the device of the invention for the treatment of skin—referred to below simply as “needle roller”—first and second needles are thus connected to different poles of a voltage source so as to establish an electric field between the differently polarized tips of the needles when they pierce the skin. First needles are arranged in a (first) row being distanced in a circumferential direction of the roller from the (second) row of second needles. Due to the distance of the rows and their connection with the different poles of the voltage source, an electrical field builds up between the differently polarized needles tips piercing the skin when the needle roller is passed over the skin. It has thus only to be ensured that both needle rows can penetrate the skin simultaneously when the roller is passed over it. With reference, therefore, to the arrangement relative to the peripheral surface of the roller, from which the needles protrude, the first row of needles is preferably immediately adjacent in a circumferential direction to the second row of needles. In this way it can be made sure that the distance between the first needles and the second needles in small enough for both rows to penetrate the skin simultaneously. Both needles rows suitably also extend parallel with respect to the longitudinal axis of the roller and also with respect to each other. However, also a slightly slanted arrangement, tilted with respect to the longitudinal roller axis, is principally possible.

The needles of a row are preferably arranged exactly linearly one after the other in the longitudinal direction of the roller axis, and especially preferably over the entire width of the roller. This is, however, not mandatory but it is also possible to make the rows shorter and/or to arrange the needles of a row in an offset or staggered manner as long as neighboring rows do not overlap. That is, first and second needles need not necessarily be arranged linearly but can be arranged within a strip extending over the width of the roller. The parallel longitudinal edges of the strip are then defined by the outermost needles of a row. The strip comprising the first needles and the strip comprising the second needles are arranged at a distance from each other. When the needles of the first and the second row penetrate the skin, on account of the moisture of the skin, there occurs migration of ions in the skin in the direction running from the end of the needles connected to the positive pole of the voltage source towards the end of the needles which are connected to the negative pole of the voltage source. The electromagnetic field builds up exclusively and specifically between the differently polarized needle rows and can be very precisely adjusted by suitably selecting the length of the needles and the spacing between the needles.

The device of the invention thus permits selective electrostimulation of the skin and ideally combines the positive effects that can be achieved in this way with the skin-stimulating action of a conventional needle roller. The electromagnetic field induced in the skin replaces the natural electromagnetic field that has ceased to exist following an injury, comparable to the use of a defibrillator after a cardiac arrest, and re-stimulates the flow of ions in the skin. Thus a breakdown of the bioelectric potentials (TEPs) is hindered, and the supply of blood and oxygen to the wound is restored such that the wound is again supplied with the endogenic substances required for wound healing. In all, the wound healing process is greatly accelerated and promoted, while at the same time the risk of scarring of the closing wound is reduced.

Another possible application of the device of the invention is the treatment of senescent skin. One cause of cutaneous senescence is that the peripheral supply of blood declines with increasing age. Deficiency of oxygen, decreasing cellular nourishment, and a reduced cleansing process are the causes of cutaneous senescence. In addition, there is a reduction in the proliferative capacity of the skin cells, more particularly the keratinocytes, for example as a result of intense exposure to solar radiation and the effect of exposure to UVA or UVB radiation, which likewise detracts from the appearance of the skin. In addition, such regenerative processes as decline with age can be promoted by treatment with the device of the invention, by means of which the action of an electromagnetic field re-activates cellular regeneration.

When connecting a plurality of first needles and second needles to the voltage source, this must not necessarily include all of the needles of a row. Rather, only specific needles from a row need be selected for connection to the voltage source. Preferably, electrical contact between the needles and the poles of the voltage source is established in such a manner that in the skin area pierced by the needles to which a potential is applied an electromagnetic field is produced in which a flow of ions predominates in a specific direction. If possible, this direction should not change or only insignificantly change during the course of the skin treatment using the device of the invention with, as far as possible, no chance of it reversing, since this might have a negative effect on the wound healing process or might at least retard it. By changing the polarity of adjacent rows of needles it is possible, for example, to produce a flow of ions in the treated skin area that takes place in the direction of advance of the needle roller over the skin, or contrary thereto. To this end, care must be taken to ensure that the skin is simultaneously pierced only by pairs of needle rows that are of consistently different polarity—for example always by a first row of needles, as regarded in the direction of advance, which is positively polarized while the second row of needles is negatively polarized—and that the skin is not pierced by further rows of needles exhibiting a different polarity such as might produce a reverse flow of ions in the skin. This can be basically achieved by a variety of measures.

In a very simple though not preferred variant, only two adjacent rows of needles, as regarded in the direction of the peripheral surface, are connected to the poles of the voltage source, while the remainder of the needles projecting from the peripheral surface of the roller are not connected thereto. However, in this way only a small and narrowly delimited electromagnetic field can be produced in the skin. A further possibility consists in ensuring, by careful adjustment of the lengths of the needles and of the spacing between the rows of needles, that in each case only the needles of two adjacent rows of consistent polarity can pierce the skin, while pairs of needle rows other than these two rows cannot do so. In this way, basically all of the rows of needles projecting from the roller can be connected to the voltage source. A disadvantage of this arrangement can be, however, that the pairs of needle rows that are adjacent to each other must be kept relatively far apart, which reduces the total number of needles projecting from the peripheral surface of the roller and thus weakens the effect of the needle roller, that is to say, makes it necessary to carry out a longer treatment to achieve the desired effect. In addition, the irregular intervals in the peripheral direction (smaller distance between the rows within a pair of needle rows, greater distance between adjacent pairs of rows) can possibly have a negative influence on the movement of the needle roller along the surface of the skin.

According to a further variant, the peripheral surface of the roller is provided with a plurality of pairs of rows of first needles and second needles that in each case are connected either to the first pole or to the second pole of the voltage source. These pairs of rows of needles connected to the different poles of the voltage source are however separated, in each case, by at least one row of needles not connected to the voltage source. Thus even when the needles in these rows that are not connected to the voltage source pierce the skin simultaneously with those rows of needles that are connected to the voltage source, this will not influence the flow of ions within the skin. When the rows of needles that follow on each other in the peripheral direction are connected to the different poles in a consistent manner (for example plus, minus, zero, plus, minus, zero etc.), the direction of the electromagnetic field in the skin will stay the same. It is self evident that within a row of needles not all of the needles need to be connected to a pole of a voltage source, but only some of them need be connected thereto, for example every other needle. This basically applies to all examples in which rows of needles are brought into contact with the voltage source.

In a particularly preferred embodiment of the invention, the first terminal, the second terminal, the first contacting zone, and the second contacting zone, by means of which the first needles and the second needles can be connected to the poles of the voltage source, are designed such that in each case only those first and second needles are connected to the voltage source that are currently piercing the skin. This can be achieved by designing the first terminal and the first contacting zone and also the second terminal and the second contacting zone such that they can, respectively, be releasably connected to each other. An electric contact is established, in each case, only when the corresponding needles have been moved, as a result of the rotation of the roller, to a position in which they can pierce the skin. Thus it is particularly preferred when contact can be established between the respective pair comprising a terminal and a contacting zone by means of the rotation of the roller about its longitudinal axis and subsequently released from said contact. This can be achieved, for example, when the terminals and contacting zones are in the form of sliding contacts. More specifically, the first terminal to be connected to the first pole of the voltage source is oriented relatively to the first contacting zone that is electrically connected to the first needles so that the two surfaces of contact come into contact with each other when the roller is rolled across the skin such that the first needles point in the direction towards the skin. The same applies with respect to the second terminal, the second contacting zone, and the second needles electrically connected thereto. Thus as soon as the first and second needles pierce the skin, the respective associated sliding contacts come into contact with each other, current begins to flow, and between the first needles and the second needles there is formed an electromagnetic field, which in turn induces a flow of ions between the first and second needles in the skin. Further rotation of the roller causes the first and second needles to be withdrawn from the skin, while the sliding contacts lose contact with each other and the flow of current stops. This method of establishing contact makes it possible to connect all of the rows of needles in the peripheral direction of the roller, even when these are relatively narrowly spaced from each other, without any unintentional reversals of direction occurring in the electromagnetic field induced in the skin when more than two adjacent rows of needles pierce the skin.

The first and second contacting zones serving to establish contact between the terminals connected to the voltage source and the first and second needles can basically be led out to an arbitrary site on the surface of the roller in order to make it possible to establish contact. It is particularly preferred that the first and second contacting zones be located on an end face of the roller, and more preferably they are located on opposite end faces of the roller. In this way, the two contacting zones are kept far apart from each other, and there is sufficient room for each of them to establish contact. The first and second terminals serving to provide an electrical connection between the contacting zones and the voltage source are situated so as to correspond to the positions of the associated contacting zones. If the latter are located on an end face of the roller, it is preferred that the terminals be disposed on an inside surface of a bracket holding the roller. If the first and second contacting zones are located on opposite faces of the roller, the first and second terminals will correspondingly by situated at opposite inside surfaces of the bracket. In order to ensure that a good contact is made between the associated pairs comprising terminal and contacting zone, at least one of the two paired components can be spring-biased in the direction of the other component.

In the case of the arrangement of needles in consecutive rows, as is described above, it is preferred that several or all of the first needles are connected in series with the first contacting zone and several or all of the second needles are connected in series with the second contacting zone. It is particularly preferred that each of the contactable rows of needles has both a first contacting zone for establishing contact with the first terminal and a second contacting zone for establishing contact with the second terminal. Depending on the position of the row of needles with reference to the first and the second terminal, the row of needles can for this reason function, on the one hand, as the first row of needles when it is in contact with the first pole of the voltage source, or, on the other hand, as the second row of needles, when contact is established with the second pole of the voltage source. Depending on its position, i.e. on the angular position of the roller, a row of needles can accordingly be connected either to the positive or to the negative pole of the voltage source. That is to say, the rotation of the roller changes the polarity of the needles in a row of needles from, for example, positive to negative and then to “disconnected”.

The establishment of contact between the electrically conducting interconnected parts of the device of the invention can basically take place in any desired manner, for example, with the aid of electrically conducting wires. This manner of establishing contact is advantageously used for electrically connecting the first and second terminals to the poles of the voltage source, it being preferred to lead the wire through the interior of the bracket and out through the rear end of the fork when the voltage source used is an external voltage source located away from the needle roller. However, it is basically possible to integrate the voltage source in the needle roller, for example in the form of a battery or an accumulator. In this case, the needles or rows of needles may be directly connected to the poles of the voltage source. The contacting zones are then, for example, the terminal bases of the needles (i.e. those ends of the needles that are opposite the tips of the needles projecting from the periphery of the roller). The contacting zones are in this case integral components of the needles: i.e. no separate connecting means are required for establishing electrical contact between the contacting zones and the needles. Advantageously, a switch is provided for switching the voltage source on and off when such an integrated voltage source is used, this being preferably disposed in the interior of the roller. In the case of a non-rechargeable voltage source, the device of the invention is a throw-away product, which is discarded when the energy stored in the voltage source has been consumed.

However, preference is given to devices in which the voltage source is in the form of an external voltage source situated away from the needle roller. Preferably, it is a direct current source capable of delivering either a linear and/or a pulsed direct current. Very advantageously, the voltage source is in the form of a variable voltage source allowing for adjustment of the voltage input. This will make it possible for the operator to selectively adjust, say, the size of the inputted voltage, the size of the inputted current, the type of inputted current (pulsed or linear), and also the polarity. Particularly preferred is a voltage source that is configured to deliver a direct current voltage in the two to four digit millivolt range, more particularly in the two to three digit millivolt range. The current strength will usually be in the milliampere range.

For the purpose of establishing contact between the first contacting zone and the first needles and between the second contacting zone and the second needles, use can likewise be made of any desired electrical conductor, such as wires, for example. In this case it is preferred, however, to establish the electrical contact with the aid of electrically conductive plastics materials. The electrically conductive plastics material can be a conductive adhesive, which can at the same time serve to attach the needles to the roller. More preferably, at least the rear ends of the needles below the peripheral surface of the roller can be embedded in the conductive plastics material.

As known from the conventional “non-electric” needle rollers, the needles can be embedded in needle carriers made of plastics material. In this case, use is made of, for example, needle carriers which have a substantially pie-shaped cross-section in a direction at right angles to the longitudinal axis of the roller and which in each case hold a row of needles extending in the transverse direction of the roller parallel to its longitudinal axis. A plurality of such pie-shaped needle carriers are then joined together in order to form the complete roller. Such pie-shaped needle carriers can likewise be used for the production of the electrified device of the invention. Preferably, the needle carriers then consist at least partially of a conductive plastics material that establishes electrical contact of the individual needles with each other. However, in order to prevent short circuits from occurring between the individual needle carriers and the needles embedded therein, the respective needle carriers must be insulated from each other. Preferably, this is achieved in that the needle carrier is placed in a support made of a non-conducting material, more particularly of a non-conducting plastics material, which is configured such that it prevents direct contact between the individual needle carriers. Advantageously, this support therefore has a star-shaped cross-section oriented at right angles to the longitudinal axis of the roller. The needle carriers are placed between the individual beams of the star. More preferably, this is achieved by causing the needle carriers to be held in the support merely by means of a force fit not requiring any further fixing means. Alternatively or additionally, the needle carriers may, however, be attached adhesively to the support.

If all of the needle carriers consist of an electrically conductive plastics material, it may be advantageous to insulate their surfaces that come into contact with the skin This can be achieved, for example, by applying a non-conducting coating to that surface of the needle carrier which forms the peripheral surface of the roller. In this respect, the said material may be in the form of a layer of non-conducting plastics film, for example a shrink film. If desired, those projecting regions of the needles that are directly adjacent to the peripheral surface of the roller may also be insulated, for example likewise by means of a non-conducting covering layer, so that only the extreme tips of the needles are electrically conducting and only in these regions can the electromagnetic field form in the skin. For the purpose of protection and the prevention of allergic reactions, the needles may be coated at least in those regions thereof by means of which they pierce the skin. The protective coating may be of, for example, gold, silver, titanium or some other inert metal, or mixtures thereof or alloys thereof.

As mentioned above, the device of the invention may be basically constructed in substantially the same way as the needle roller described in DE 10063634 A1. When applying the invention to the device described in EP 1764129 A1, one or more of the rollers can be provided with means for establishing electrical contact. Since the construction of the needle roller is basically known, there is no need to describe it here in detail. Thus it is only necessary to provide a brief description of some characteristics and dimensions, which are also of significance to the present invention.

Preferably, the device of the invention accordingly possesses at least one of the following characteristics:
from 8 to 30, preferably from 12 to 24 and more preferably from 16 to 20 needles in the peripheral direction of the roller,
from 2 to 16, preferably from 3 to 12 and more preferably from 4 to 9 needles per row,
a free length of the needles above the peripheral surface of from 0.1 to 3.0 mm, preferably from 0.2 to 2.5 mm and more preferably from 0.25 to 2.0 mm,
a maximum diameter of the needles in the region thereof extending above the peripheral surface of from 0.05 to 0.3 mm and preferably from 0.08 to 0.2 mm, the needle ends are tapered towards the tips of the needles and are more particularly machine ground in the region of the tip of each needle,
the distance between the tips of the first and second needles located in two adjacent rows of needles directly opposite each other is from 2.5 to 5 mm, preferably from 3.0 to 4.5 mm and more preferably from 3.5 to 4.0 mm.

### BRIEF DESCRIPTION OF THE DRAWINGS

The invention will be explained in greater detail below with reference to an exemplary embodiment illustrated in the drawings, in which like parts are designated by like reference signs and in which:

FIG. 1a is a schematic illustration of a side view of a device of the invention for the treatment of skin;

FIG. 1b is a schematic top view of the device of the invention as shown in FIG. 1a but rotated through 90°;

FIG. 2a is a schematic top view of a device of the invention for clarification of the electrical means for establishing contact;

FIG. 2b shows schematically the device as illustrated in FIG. 2a but rotated through 90°;

FIG. 3a schematically shows a side view of an end face of the roller as shown in FIG. 2a;

FIG. 3b schematically shows the roller as shown in FIG. 3a but rotated through 90°;

FIG. 4 schematically shows a further top view of the end face of a roller mounted on a bracket;

FIG. 5a schematically shows a cross-sectional view of a roller,

FIG. 5b schematically illustrates a cross-sectional view of the support used in the roller as shown in FIG. 5a;

FIG. 5c schematically shows a needle carrier for the roller as shown in FIG. 5a as a perspective view; and

FIG. 5d schematically shows a cross-section of the needle carrier as shown in FIG. 5a.

### DETAILED DESCRIPTION OF PREFERRED EMBODIMENTS

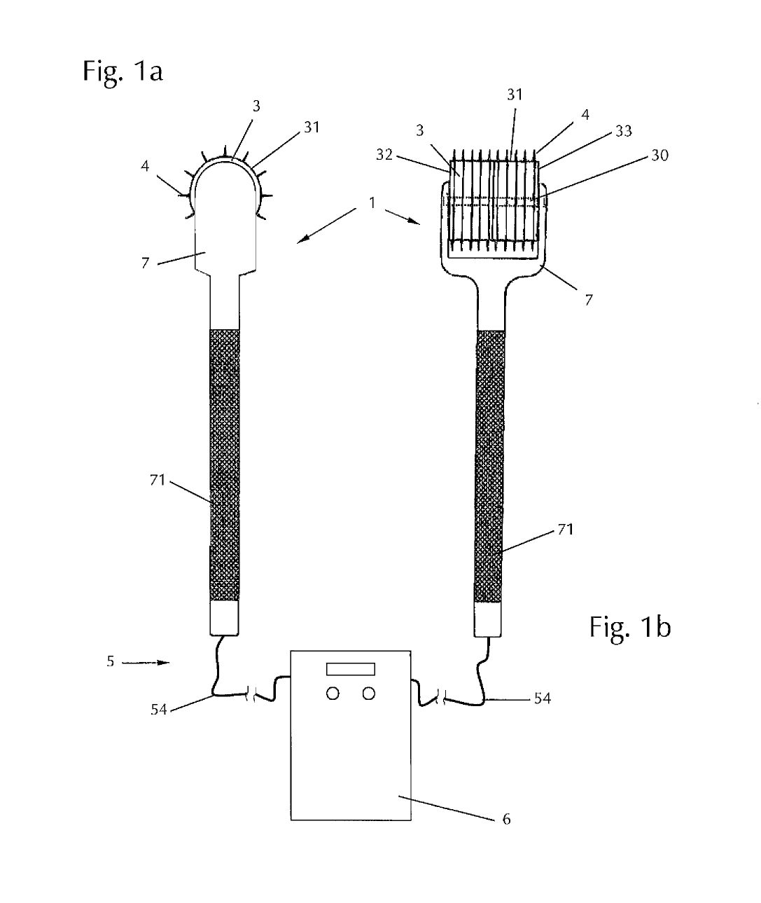

FIGS. 1a and 1b show a perspective view of an exemplary embodiment of a device of the invention 1 comprising a needle roller, which is connected to a voltage source 6. The basic construction of the needle roller is substantially the same as that of the roller that has already been described in DE 10063634 A1. The needle roller comprises a roller 3, which is mounted on a bracket 7 for rotation about its longitudinal axis 30. A number of needles 4 project radially outwardly from the peripheral surface 31. In addition to the needle roller described in DE '634, the device of the invention 1 comprises means 5 for establishing electrical contact between at least two rows of the needles 4 and the voltage source 6. The connection to the voltage source 6 is established in this case via a current lead 54, which is guided from the roller 3 via the bracket 7 and through the handle 72 connected to the bracket 7 and out at the bottom end thereof towards the voltage source 6. The voltage source 6 is a variable voltage source, by means of which a linear or pulsed direct current can be applied to the contacted needles 4. The voltage strength and current strength can be adjusted by the user as desired according to the requirements necessary for the skin treatment.

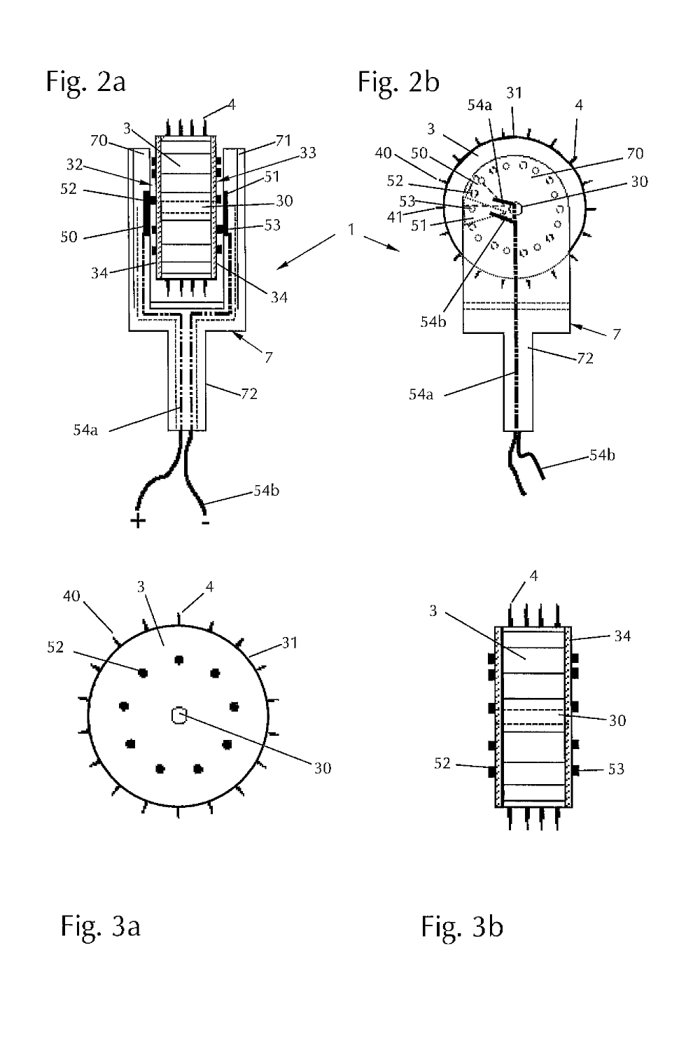

The method of establishing contact between the voltage source 6 and the needles 4 is explained in greater detail below with reference to the figures. FIG. 2a is a top view of the head region of a device of the invention 1, more specifically a top view of the peripheral surface of a roller 3 mounted on a bracket 7. FIG. 2b shows the device as shown in FIG. 2a, rotated through 90°, i.e. it is a top view of the end face of the roller 3 supported by the bracket 7. The establishment of the electrical contact with the voltage source 6 is effected, as already discussed with reference to FIGS. 1a and 1b, via a current lead 54, which is made up of a supply line 54a connected to the positive pole of the voltage source 6, and a supply line 54b connected to the negative pole of the voltage source 6. The two supply lines 54a and 54b are first of all guided through the handle 72 of the bracket 7 and then separated in the forked end, the wire 54a passing in the direction of the left-hand fork end 70, as illustrated in the figure, while the wire 54b is guided towards the right-hand end 71 of the bracket, as illustrated in the figure. The wire 54a is connected in the region of the fork end 70 to a first terminal 50, which is guided outwardly towards the inside surface of the fork region 70 and comprises a surface of contact in the direction towards the roller 3. The second wire 54b is connected in the same manner to a terminal 51 in the region of the right-hand fork end 71.

Via the terminal 50 there is established an electrically conducting contact with a contacting zone 52, which outwardly projects from the left-hand end face 32 of the roller 3, as depicted in the figure. In order to ensure that there is a sufficiently good contact between the terminal 50 and the contacting zone 52, the terminal 50 can be mounted with a spring bias such that it is pressed against the contacting zone 52 with adequate pressure. The contacting zone 52 is the end of a contact pin projecting beyond the end face of the roller 3, which contact pin extends through an end cap 34, which covers the end face 32, into the roller 3, where it is electrically connected to an associated row of needles 4. The same row of needles is connected in the same way to a second contacting zone 53 that protrudes beyond the right-hand end face 33 of the roller 3, as depicted in the figure. In the embodiment shown, a row consists in each case of four needles 4. Each of the total of 18 rows of needles possesses a first and a second contacting zone 52, 53. The establishment of electrical contact between the contact pins and the needles is described in greater detail below.

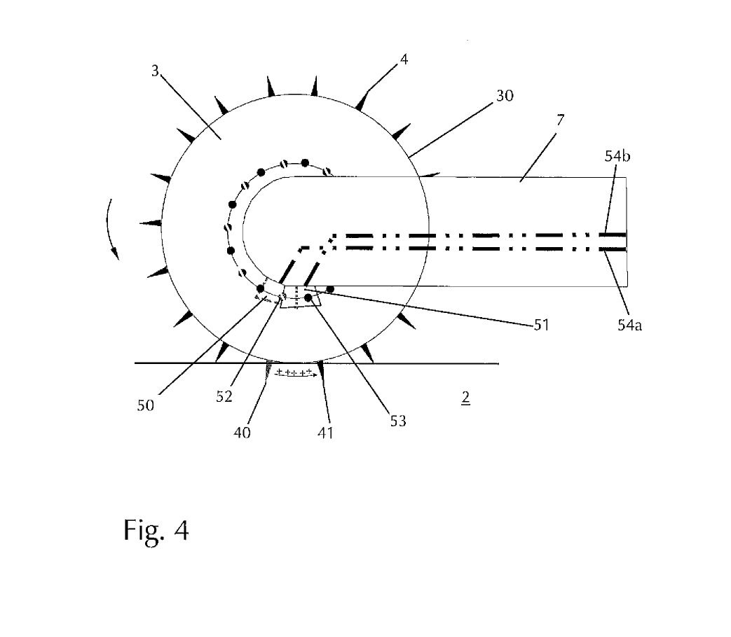

The making and breaking of electrical contacts between the terminal 50 and contacting zone 52, on the one hand, and between the terminal 51, which can also be spring-biased, and the contacting zone 53, on the other hand, is effected by the rotation of the roller 3 about its longitudinal axis 30. As shown in FIG. 2b, the terminals 50 and 51, which are located on opposite sides of the fork ends 70 and 71, are staggered in the peripheral direction of the roller. As a result, the first terminal 50 comes into contact with a first contacting zone 52 of a first row of needles 40, and the second terminal 51, however, comes into contact with the second contacting zone 53 of a second row of needles 41 that is adjacent to the first row of needles 40. The first row of needles 40 is thus positively charged, while the adjacent row of needles 41 is negatively charged. The position of the terminals 50 and 51 is chosen such that the two contacted rows of needles 40 and 41 pierce the skin when the needle roller is placed on the skin 2 (cf. FIG. 4). On account of the different polarities of the first and second rows of needles, a flow of ions is induced in the skin from positively charged needles 40 towards the negatively charged needles 41 to stimulate wound healing by electrostimulation.

When the device 1 is advanced along the surface of the skin (in FIG. 4 in the direction towards the left-hand side of the sheet), the roller 3 rotates about the longitudinal axis 30 in the anticlockwise direction (as indicated by the arrow). The rotation of the roller 3 causes the contacting zones 52 and 53 to move further in the anticlockwise direction and thus away from the terminals 50 and 51 which are mounted on the bracket 7 and with which they had previously been in contact. It is being assumed that the roller 3 is rotated just sufficiently as to move the first contacting zone 52 to the previous position of the second contacting zone 53. In this way, the previously positively charged first needles 40 now become negatively charged via the second terminal 51 connected to the negative pole of the voltage source 6. They thus become second needles 41 due to the advancement of the roller. The second row of needles previously connected to the negative pole, however, has now left the second contacting zone 53 and is therefore no longer connected to the voltage source 6. Instead, the next—in the figure the left-hand—adjacent row of needles now moves into the region of the first terminal 50, this row of needles now being positively charged on account of its contacting zone 52 being connected to the terminal 50. Thus, an electromagnetic field is again induced in the skin between the adjacent and differently charged pairs of needle rows, which electromagnetic field flows in the same direction as the previous one. Thus, when the needle roller is advanced, there is no change in the direction of flow of ions in the skin, and the ion flow retains its strength and direction. There is thus produced a constant electromagnetic field in the skin irrespective of the angular position of the device of the invention. If, during a skin treatment, it should be desired to effect reorientation of this electromagnetic field, this can be achieved by reversing the polarity at the voltage source 6,

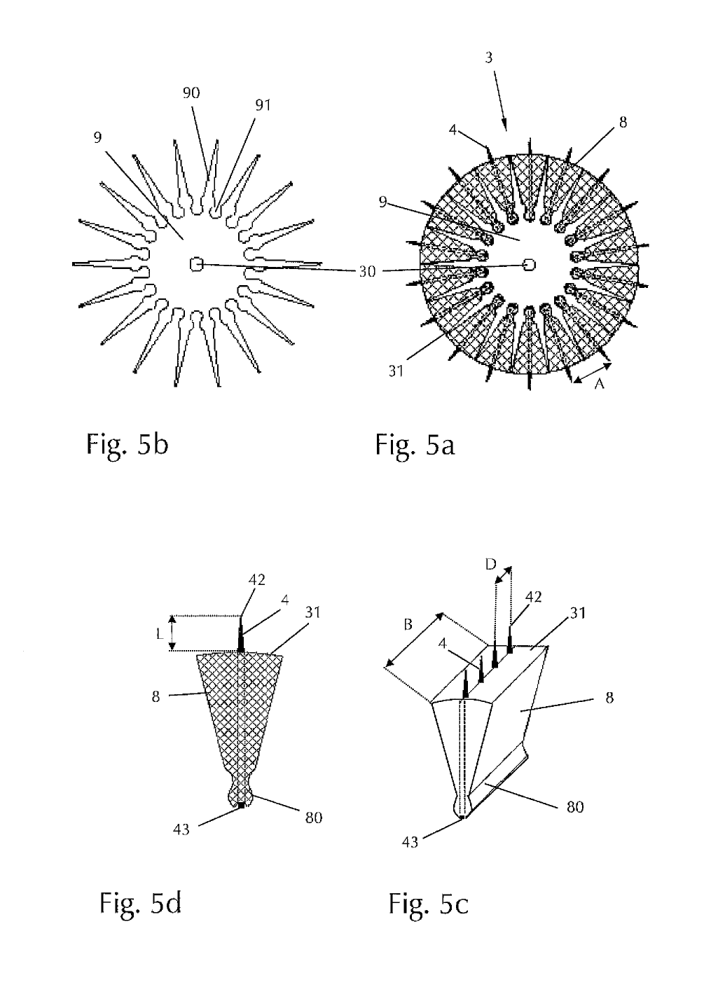

FIGS. 5a to 5d serve to illustrate the interior structure of the roller 3 and the means of establishing contact between the individual needles and the contacting zones 52 and/or 53. As shown in FIG. 5a, the needles 4 are embedded in individual needle carriers 8, which have a substantially pie-shaped cross-section in the sectional view oriented at right angles to the longitudinal axis 30 of the roller 3. Each of the needle carriers 8 extends substantially across the entire width of the roller 3. Only the end faces are additionally covered by end caps 34. Each of the needle carriers 8 accommodates a row of needles 4, and, in the present embodiment, each row consists of four individual needles, which are embedded in the needle carrier and are spaced from each other at equal intervals D of in this case approximately 2 mm. The width B of the needle carrier 8 is approximately 8 mm, and the total width including the mounted end caps is approximately 10 mm. For example, the four needles 4 of a row of needles are potted in a needle carrier 8 of plastics material. Their tips project from the external periphery 31 of the needle carrier 8 or the roller 3 over a distance (length L) of, say, from 0.1 to 3 mm. The needles 4 are at least in this projecting region, outwardly tapered towards the tip 42 of the needle and are preferably provided with a machine-ground surface. The maximum diameter of the needles 4 in the region thereof projecting above the peripheral surface 31 is advantageously between 0.05 and 0.3 mm and more preferably between 0.08 and 0.2 mm. Usually, the maximum diameter of the needles in this projecting region of length L will be located in the region of the needles 4 which is directly adjacent to the peripheral surface 31. The distance A between the tips of the needles in two directly adjacent rows of needles is approximately 3.5 mm, in the example shown.

In the case illustrated, the roller 3 comprises eighteen rows of needles and thus possesses eighteen needle carriers 8. These are anchored in a support 9, which is illustrated in FIG. 5b in the cross-section oriented at right angles to the longitudinal axis 30 of the roller 3. The support 9 is of plastics material and has a substantially star-shaped cross-section having eighteen beams 90, between which the needle carriers 8 are individually force fitted. The needle carriers 8 are held by a force fit between two beams 90, wherein the drop-shaped bottom regions 80 engage corresponding spherical recesses 91 in the support 9 to form a type of locking joint, thus ensuring a firm anchorage of the needle carrier 8 in the support 9. If desired, the needle carriers 8 can alternatively be adhesively held in position in the support 9.

There are various ways of establishing electrical contact of the needles disposed in a row and fixed in a needle carrier 8. One such method consists in interconnecting the needles in the region of their terminal bases 43 by means of a conductive adhesive or a wire or by a similar method. In this case, however, the ability to use material for the needle carrier 8 that is conductive has been chosen. For this purpose it can be basically sufficient to produce only a sub-region of the needle carrier 8, for example the bottom region 80, from the conductive material. In the present case, however, the needle carrier is made entirely of a conductive plastics material, which ensures that all of the needles will be electrically interconnected and also electrically connected to the relevant contacting zones 52 and 53. The contacting zones 52 and 53, which, as illustrated in FIG. 3b, for example, are located on the different end faces of every needle carrier 8, are here in the form of contact nipples, which are guided through matching bores in the end caps 34 and are fixed, for example by adhesion, by way of their inside surfaces to a respective face of the needle carrier 8, and are electrically connected to the electrically conductive needle carrier 8 and/or to the needles otherwise electrically interconnected. As described above, depending on the rotary position of the roller 3 about its longitudinal axis 30, either the first contacting zone 52 will come into electrically conductive contact with the first terminal 50 or the second contacting zone 53 with the second terminal 51. By this means, the needles 4 will be connected either to the positive pole or to the negative pole of the voltage source 6 and be charged accordingly. Short circuits between the adjacent needle carriers 8 and the rows of needles that are adjacent to each other are avoided due to the fact that the material of the support 9 is nonconductive and the individual needle carriers 8 are thus insulated from each other even when they are all made of a conductive material,

### LITERATURE

1. Foulds L., Barker A. Human skin battery potentials and their possible role in wound healing. Br J Dermatol 1983; 109:515-22.
2. L. C. Kloth, Electrical Stimulation for Wound Healing: A Review of Evidence From In Vitro Studies, Animal Experiments, and Clinical Trials. 2005 Sage Publications.
3. Jaffe L., Vanable J. Electrical fields and wound healing. Clin Dermatol 1984; 2(3):34-44.
4. Cheng K., Tarjan P., Oliveira-Gandia M., et al. Anocclusive dressing can sustain natural electrical potential of wounds. J Invest Dermatol 1995; 104(4):662-5.
5. Eltinge E., Cragoe E. Jr., Vanable J. Jr. Effects of amiloride analogues on adult Notophthalmus viridescens limb stump currents. Comp Biochem Physiol 1986; 89A:39-44.
6. Falanga V., Bourguignon G., Bourguignon L. Electrical stimulation increases the expression of fibroblast receptors for transforming growth factor-beta. J Invest Dermatol 1987; 88:488-92.
7. Orida N., Feldman J. Directional protrusive pseudopodial activity andmotility in macrophages induced by extra-cellular electric fields. Cell Motil 1982; 2:243-55.
8. Monguio J. Über die polare Wirkung des galvanischen Stromes auf Leukozyten. Z Biol 1933; 93:553-9.
9. Fukushima K., Senda N., Inui H., et al. Studies of galvanotaxis of leukocytes. Med J Osaka Univ 1953; 4(2-3):195-208.
10. Dineur E. Note sur la sensibilities des leukocytes a l′electricite. Bulletin Seances Soc Beige Microscopic (Bruxelles) 1891; 18:113-8.
11. Canaday D., Lee R. Scientific basis for clinical application of electric fields in soft tissue repair. In: Brighton C, Pollack S, editors. Electromagnetics in biology and medicine. San Francisco: San Francisco Press; 1991.
12. Erickson C., Nuccitelli R. Embryonic fibroblastmotility and orientation can be influenced by physiological electric fields. J Cell Biol 1984; 98:296-307.
13. Yang W., Onuma E., Hui S. Response of C3H/10T1/2 fibroblasts to an external steady electric field stimulation. Exp Cell Res 1984; 155:92-7.
14. Nishimura K., Isseroff R., Nuccitelli R. Human keratinocytes migrate to the negative pole in direct current electric fields comparable to those measured in mammalian wounds. J Cell Sci 1996; 109:199-207.
15. Sheridan D., Isseroff R., Nuccitelli R. Imposition of a physiologic DC electric field alters the migratory response of human keratinocytes on extracellular matrix molecules. J Invest Dermatol 1996; 106(4):642-6.
16. Min Zaho. Electrical fields in wound healing An overriding signal that directs cell migration. Elsevier 2008, page 674-680.
17. Min Zhao et al. Electrical signals control wound healing through phosphatidylinositol-3-OH kinase-Y and PTEN, Nature 2006, Vol 442/27.
18. Fang K., lonides E., Oster G., et al. Epidermal growth factor receptor relocalization and kinase activity are necessary for directional migration of keratinocytes in DC electric fields. J Cell Sci 1999; 112:1967-78.
19. Rowley B. Electrical current effects on E. coli growth rates. Proc Soc Exp Biol Med 1972; 139:929-34.
20. Wolcott L., Wheeler P., Hardwicke H., et al. Accelerated healing of skin ulcers by electrotherapy: preliminary clinical results. South Med J 1969; 62:795-801.
21. Deitch E., Marino A., Malakanok V., et al. Electrical augmentation of the antibacterial activity of silver nylon. Proceedings of the 3rd Annual BRAGS; 1983 Oct. 2-5; San Francisco.
22. Deitch E., Marino A., Gillespie T., et al. Silver nylon: a new antimicrobial agent. Antimicrobial Agents Chemother 1983; 23:356-9.
23. Marino A., Deitch E., Albright J. Electric silver antisepsis. IEEE Trans Biomed Eng 1985; 32(5):336-7.
24. Cohnano G., Edwards S., Barranco S. Activation of antibacterial silver coatings on surgical implants by direct current: preliminary studies in rabbits. Am J Vet Res 1980; 41(6):964-6.
25. Thibodeau E., Handelman S., Marquis R. Inhibition and killing of oral bacteria by silver ions generatedwith lowintensity direct current. J Dent Res 1978; 57:922-6.
26. Alvarez O., Mertz P., Smerbeck R., et al. The healing of superficial skin wounds is stimulated by external electrical current. J Invest Dermatol 1983; 81(2):144-8.
27. Falcone A., Spadero J Inhibitory effects of electrically activated silver material on cutaneous wound bacteria. Plast Reconstr Surg 1986; 77(3):445-58.
28. Becker R., Spadero J. Treatment of orthopedic infections with electrically generated silver ions. J Bone Joint Surg Am 1978; 60(7):871-81.
29. Junger M., Zuder D., Steins A., et al. Treatment of venous ulcers with low frequency pulsed current (Dermapulse): effects on cutaneous microcirculation. Der Hautarzt 1997; 18:879-903.

What is claimed is:

1. A device for the treatment of skin, comprising a roller which is mounted for rotation about its longitudinal axis and on a peripheral surface of which a number of needles protrudes radially outwardly, and comprising means for establishing electrical contact between at least part of said needles and a voltage source, comprising
a first terminal that is capable of being electrically connected to a first pole of said voltage source,
a second terminal that is capable of being electrically connected to a second pole of said voltage source,
a first contacting zone that is electrically connected to first needles,
a second contacting zone that is spaced from, and electrically insulated from, said first contacting zone and that is electrically connected to second needles,
said terminals and said contacting zones being configured such that when said first terminal is connected to said first contacting zone, said second terminal is connected to said second contacting zone,
wherein said first and said second needles are each arranged in a row, said rows being separated from each other in a circumferential direction of said roller.

2. The device as defined in claim 1, wherein said first and said second contacting zones are led out to a surface of the roller.

3. The device as defined in claim 2, wherein said first and said second contacting zones are led out to an end face of the roller.

4. The device as defined in claim 3, wherein said first and said second contacting zones are located on opposite end faces of said roller.

5. The device as defined in claim 3, wherein said first and said second terminals are mounted on an inside surface of a bracket holding said roller.

6. The device as defined in claim 4, wherein said first and said second terminals are mounted on opposite ends of the inside surface of the bracket.

7. The device as defined in claim 1, wherein said first terminal and said first contacting zone and said second terminal and said second contacting zone are each releasably connected.

8. The device as defined in claim 1, wherein the respective releasable electrical connection can be established or broken by rotating said roller about its longitudinal axis.

9. The device as defined in claim 1, wherein said terminals and said contacting zones are in the form of sliding contacts.

10. The device as defined in claim 1, wherein said first needles and said second needles are in each case disposed in a row extending parallel to the longitudinal axis of said roller.

11. The device as defined in claim 1, wherein said needles are embedded in a needle carrier.

12. The device as defined in claim 11, wherein said needles are embedded in a substantially pie-shaped needle carrier.

13. The device as defined in claim 12, wherein said needle carrier is made of an at least partially conductive plastics material.

14. The device as defined in claim 13, wherein said needle carriers are held by a support of a non-conducting material having a substantially star-shaped cross-section in a direction at right angles to the longitudinal axis of the roller, which support electrically insulates said needle carriers from each other.

15. The device as defined in claim 9, wherein a multitude of adjacent rows of needles are oriented in the peripheral direction of said roller.

16. The device as defined in claim 15, wherein each row of needles has both a first contacting zone and a second contacting zone at opposite ends of said row.

17. The device as defined in claim 15, wherein said terminals are configured such that rows of needles that are adjacent to each other in the peripheral direction of said roller are capable of being brought into electrical contact with said voltage source.

18. The device as defined in claim 1, possessing at least one of the following characteristics:
from 8 to 30 needles in the peripheral direction of the roller,
from 2 to 16 needles per row,
a free length of the needles above the peripheral surface of from 0.1 to 3.0 mm,
a maximum diameter of the needles in a region thereof extending above the peripheral surface of from 0.05 to 0.3 mm,
the needle ends are tapered towards the tips of the needles,
the distance between the tips of the first and second needles located in two adjacent rows of needles directly opposite each other is from 2.5 to 5 mm.

19. The device as defined in claim 1, wherein said voltage source is configured so as to supply one of a linear and a pulsed direct current voltage.

20. The device as defined in claim 19, wherein said voltage source is in the form of a variable voltage source.
